(19) United States
(12) Patent Application Publication
(10) Pub. No.: US 2025/0311995 A1
(43) Pub. Date: Oct. 9, 2025

(54) MEDICAL IMAGE DIAGNOSTIC DEVICE AND METHOD OF CONTROLLING MEDICAL IMAGE DIAGNOSTIC DEVICE

(71) Applicant: CANON MEDICAL SYSTEMS CORPORATION, Otawara-shi (JP)

(72) Inventor: Ryusei SAIKI, Nasushiobara (JP)

(73) Assignee: CANON MEDICAL SYSTEMS CORPORATION, Otawara-shi (JP)

(21) Appl. No.: 19/095,156

(22) Filed: Mar. 31, 2025

(30) Foreign Application Priority Data
Apr. 5, 2024 (JP) 2024-061665

(51) Int. Cl. A61B 6/00; A61B 6/03

(52) U.S. Cl. CPC A61B 6/547; A61B 6/032; A61B 6/5205

(57) ABSTRACT

A medical image diagnostic device of an embodiment includes an imaging unit and processing circuitry. The imaging unit is configured to image a subject at a plurality of different positions including a predetermined reference position while rotating around the subject and to generate a plurality of pieces of raw image data including information indicating the predetermined reference position. The processing circuitry is configured to correct the reference position depending on a deviation between an orientation of the subject and a direction from the subject to the predetermined reference position, to cause the imaging unit to image the subject based on the corrected reference position, and to generate a reconstructed image by reconstructing the plurality of pieces of raw image data generated by the imaging unit based on the corrected reference position.

### CROSS-REFERENCE TO RELATED APPLICATION

The present application claims priority based on Japanese Patent Application No. 2024-061665 filed Apr. 5, 2024, the content of which is incorporated herein by reference.

### FIELD

Embodiments disclosed in this specification and drawings relate to a medical image diagnostic device and a method of controlling the medical image diagnostic device.

### BACKGROUND

There is an X-ray computed tomography (CT) apparatus that captures images while rotating a pair of an X-ray tube and an X-ray detector at a high speed around a subject, and reconstructs a plurality of pieces of projection data obtained to generate a tomographic image of the subject. Conventional X-ray CT devices reconstruct images using a plurality of pieces of projection data collected in accordance with a specific position inside a gantry (for example, a reference position of an X-ray tube). Hereinafter, an image obtained by reconstruction is referred to as a “reconstructed image.” FIG. 9 is a schematic diagram showing a direction of a reference position of a conventional X-ray CT device and an orientation of a reconstructed image. As shown in FIG. 9, the conventional X-ray CT device generally capture images of a subject in a lying position on a top plate of a bed device. Therefore, the conventional X-ray CT device basically reconstructs an image such that the upward direction of the gantry (i.e., the 0-degrees direction of a rotating part inside the gantry) becomes the upward direction of the reconstructed image.

Recent X-ray CT devices include universal X-ray CT devices that can move a gantry up, down, left, right, and diagonally. FIG. 10 is a schematic diagram showing an example of a direction of a reference position of a universal X-ray CT device and an orientation of a reconstructed image. As shown in FIG. 10, the universal X-ray CT device can also capture images of subjects in a standing or sitting position. Compared to capturing images in a lying position, subjects can easily change their body orientations when images are captured in a standing or sitting position, and thus a misalignment is likely to occur between the orientation of the body of a subject in a reconstructed image and the direction of a reference position.

FIG. 11 is a diagram showing an example of a reconstructed image when capturing an image in a lying position and a reconstructed image when capturing an image in a standing position. For example, when capturing images in a standing position as shown in FIG. 11, if the orientation of the body of the subject in a reconstructed image is misaligned with the direction of the reference position, it may be difficult to perform a diagnosis such as interpretation. For this reason, it is desirable to correct such a misalignment by performing image processing to rotate the orientation of the reconstructed image. FIG. 12 is a diagram showing an example of processing of rotating a reconstructed image.

However, when processing of rotating an image has been performed, image noise such as blurred contours can occur depending on the rotation angle, in general. Further, the need for such image processing also poses the problem of increasing the time required to reconstruct an image. Furthermore, when noise reduction processing using artificial intelligence (AI) is performed, for example, the noise reduction effect can be reduced if a reconstructed image in which the orientation of the body of a subject is different from a predetermined direction is input.

### BRIEF DESCRIPTION OF THE DRAWINGS

FIG. 1 is a configuration diagram of an X-ray CT device 1 according to an embodiment.

FIG. 2 is a diagram showing the external appearance of the X-ray CT device 1.

FIG. 3 is a perspective view of the X-ray CT device 1 that examines a subject P in a lying position.

FIG. 4 is a perspective view of the X-ray CT device 1 that examines a subject P in a standing position.

FIG. 5 is a flowchart showing an operation of the X-ray CT device 1 in a first embodiment.

FIG. 6 is a flowchart showing an operation of the X-ray CT device 1 in a second embodiment.

FIG. 7 is a flowchart showing an operation of the X-ray CT device 1 in a third embodiment.

FIG. 8 is a flowchart showing an operation of the X-ray CT device 1 in a fourth embodiment.

FIG. 9 is a schematic diagram showing a direction of a reference position in a conventional X-ray CT device and an orientation of a reconstructed image.

FIG. 10 is a schematic diagram showing an example of a direction of a reference position of a universal X-ray CT device and an orientation of a reconstructed image.

FIG. 11 is a diagram showing an example of a reconstructed image when capturing an image in a lying position and a reconstructed image when capturing an image in a standing position.

FIG. 12 is a diagram showing an example of processing of rotating a reconstructed image.

### DETAILED DESCRIPTION

Hereinafter, a medical image diagnostic device and a method of controlling the medical image diagnostic device according to an embodiment will be described with reference to the drawings. An X-ray CT device which will be described below is an example of the medical image diagnostic device of the present invention. However, the medical image diagnostic device of the present invention is not limited to an X-ray CT device, and may be any other apparatus as long as it is an apparatus that captures images while rotating an imaging unit around a subject.

A medical image diagnostic device of an embodiment includes an imaging unit and processing circuitry. The imaging unit is configured to image a subject at a plurality of different positions including a predetermined reference position while rotating around the subject and to generate a plurality of pieces of raw image data including information indicating the predetermined reference position. The processing circuitry is configured to correct the reference position depending on a deviation between an orientation of the subject and a direction from the subject to the predetermined reference position, to cause the imaging unit to image the subject based on the corrected reference position, and to generate a reconstructed image by reconstructing the plurality of pieces of raw image data generated by the imaging unit based on the corrected reference position.

An X-ray CT device is a medical apparatus that includes a gantry with an opening through which a subject can be inserted, a gantry driving device that moves the gantry relative to the subject, and a support on which the subject is placed. The gantry driving device can move the gantry up, down, left, right, and diagonally, and can scan the subject in any of lying, standing, and sitting positions. The X-ray CT device is, for example, a universal X-ray CT device.

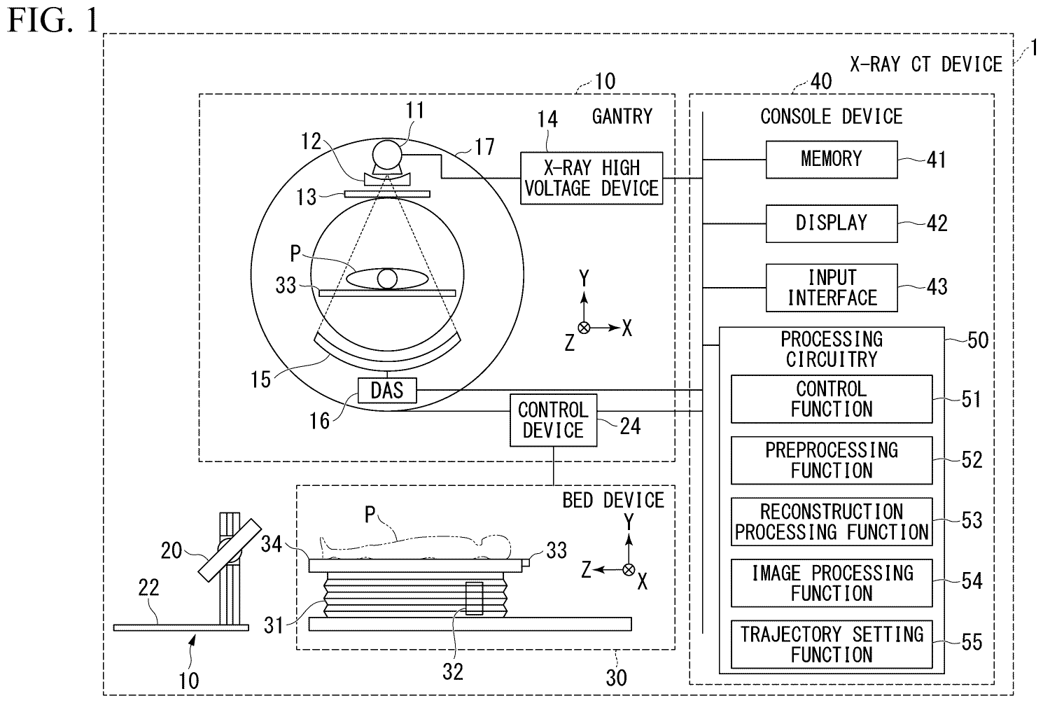

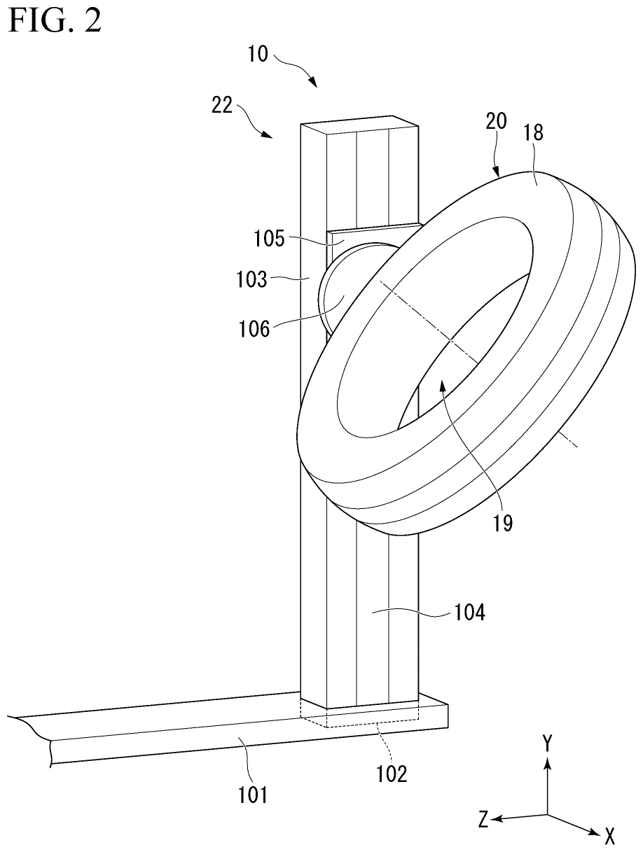

FIG. 1 is a configuration diagram of an X-ray CT device 1 according to an embodiment. FIG. 2 is a diagram showing the external appearance of the X-ray CT device 1. The X-ray CT device 1 includes, for example, a gantry 10, a bed device 30, and a console device 40. For convenience of description, FIG. 1 shows both a view of the gantry 10 in a Z-axis direction and a view of the gantry 10 in an X-axis direction, but in reality, there is only one gantry 10. In the embodiment, in a non-tilted state, a rotation axis of a rotating frame 17 or the longitudinal direction of a top plate 33 of the bed device 30 aligned in the horizontal direction is defined as the Z-axis direction (front-to-back direction), an axis that is perpendicular to the Z-axis direction and horizontal to the floor surface is defined as the X-axis direction, and a direction that is perpendicular to the Z-axis direction and orthogonal to the floor surface is defined as a Y-axis direction (up-down direction).

The gantry 10 in the X-ray CT device 1 includes, for example, a gantry 20, a gantry driving device 22, and a control device 24. The gantry 20 is supported by the gantry driving device 22. The gantry driving device 22 can move the gantry 20 in the up, down, left, and right directions, and can tilt the gantry 20 to change the orientation of the gantry 20. The control device 24 controls the operation of the gantry driving device 22.

The gantry 20 includes an X-ray tube 11, a wedge 12, a collimator 13, an X-ray high voltage device 14, an X-ray detector 15, a data acquisition system (DAS) 16, a rotating frame 17, and a cover 18. The X-ray tube 11, the wedge 12, the collimator 13, the X-ray high voltage device 14, the X-ray detector 15, the DAS 16, and the rotating frame 17 are accommodated in the cover 18.

The X-ray tube 11 generates X-rays by radiating thermoelectrons from a cathode (filament) to an anode (target) by applying a high voltage from the X-ray high voltage device 14. The X-ray tube 11 radiates X-rays to a subject P. The X-ray tube 11 includes a vacuum tube. For example, the X-ray tube 11 is a rotating anode type X-ray tube that generates X-rays by radiating thermoelectrons to a rotating anode.

The wedge 12 is a filter for adjusting the amount of X-rays (radiation amount) radiated from the X-ray tube 11 to the subject (imaged object) P. The wedge 12 attenuates X-rays that pass through the wedge 12 such that the distribution of the amount of X-rays radiated from the X-ray tube 11 to the subject P becomes a predetermined distribution. The wedge 12 is also called a wedge filter or a bow-tie filter. The wedge 12 is, for example, made of aluminum processed to have a predetermined target angle and a predetermined thickness.

The collimator 13 is a mechanism for narrowing the radiation range of X-rays that have passed through the wedge 12. The collimator 13 narrows the radiation range of X-rays by, for example, forming a slit by combining a plurality of lead plates. The collimator 13 is sometimes called an X-ray aperture. The collimator 13 may be an active collimator whose narrowing range can be mechanically driven.

The X-ray high voltage device 14 has, for example, a high voltage generator and an X-ray control device. The high voltage generator has an electric circuit including a transformer and a rectifier, and generates a high voltage to be applied to the X-ray tube 11. The X-ray control device controls the output voltage of the high voltage generator depending on the amount of X-rays to be generated by the X-ray tube 11. The high voltage generator may boost a voltage using the aforementioned transformer or boost a voltage using an inverter. The X-ray high voltage device 14 may be provided on the rotating frame 17 or on the side of a fixed frame (not shown) of the gantry 10.

The X-ray detector 15 detects the intensity of the X-rays generated by the X-ray tube 11 and incident after passing through the subject P. The X-ray detector 15 outputs an electrical signal (which may be an optical signal or the like) corresponding to the detected intensity of the X-rays to the DAS 16. The X-ray detector 15 has, for example, a plurality of X-ray detection element rows. Each of the plurality of X-ray detection element rows has a plurality of X-ray detection elements arranged in a channel direction along an arc having the focal point of the X-ray tube 11 as a center. The plurality of X-ray detection element rows are arranged in a slice direction (row direction).

The X-ray detector 15 is an indirect type detector having, for example, a grid, a scintillator array, and a photosensor array. The scintillator array has a plurality of scintillators. Each scintillator has a scintillator crystal. The scintillator crystal emits an amount of light corresponding to the intensity of incident X-ray. The grid is disposed on the surface of the scintillator array on which X-rays are incident, and has an X-ray shielding plate having a function of absorbing scattered X-rays. The grid may also be called a collimator (a one-dimensional collimator or a two-dimensional collimator). The optical sensor array has an optical sensor such as a photomultiplier tube (PMT). The optical sensor array outputs an electrical signal according to the amount of light emitted by scintillators. The X-ray detector 15 may be a direct conversion type detector having a semiconductor element that converts incident X-rays into an electrical signal.

The DAS 16 includes, for example, an amplifier, an integrator, and an analog-to-digital (A/D) converter. The amplifier amplifies an electrical signal output by each X-ray detection element of the X-ray detector 15. The integrator integrates the amplified electrical signal over a view period (which will be described later). The A/D converter converts an electrical signal indicating the integration result into a digital signal. The DAS 16 outputs detection data based on the digital signal to the console device 40. The detection data is a digital value of X-ray intensity identified by a channel number and a row number of an X-ray detection element that is a generation source, and a view number indicating a collected view. A view number is a number that changes according to the rotation of the rotating frame 17, and is, for example, a number that is incremented according to the rotation of the rotating frame 17. Therefore, the view number is information that indicates the rotation angle of the X-ray tube 11. A view period is a period that falls between a rotation angle corresponding to a certain view number and a rotation angle corresponding to the next view number.

The DAS 16 may detect view change by a timing signal input from the control device 24, an internal timer, or a signal obtained from a sensor (not shown). When full scanning is performed, if X-rays are continuously emitted by the X-ray tube 11, the DAS 16 collects a detection data group for the entire circumference (360 degrees). When half scanning is performed, if X-rays are continuously emitted by the X-ray tube 11, the DAS 16 collects detection data for half the circumference (180 degrees).

The rotating frame 17 is an annular member that supports the X-ray tube 11, the wedge 12, the collimator 13, and the X-ray detector 15 while facing them. The rotating frame 17 is supported by a fixed frame to be freely rotatable around the subject P introduced thereinside. The rotating frame 17 also supports the DAS 16. Detection data output by the DAS 16 is transmitted through optical communication from a transmitter having a light emitting diode (LED) provided on the rotating frame 17 to a receiver having a photodiode provided on a non-rotating part (e.g., the fixed frame) of the gantry 10, and transferred to the console device 40 by the receiver. The method of transmitting the detection data from the rotating frame 17 to the non-rotating part is not limited to the aforementioned method using optical communication, and any non-contact transmission method may be adopted. The rotating frame 17 is not limited to a ring-shaped member, and may be an arm-like member as long as it can support and rotate the X-ray tube 11 and the like.

The cover 18 is provided with a central opening 19. The central opening 19 is an opening into which the subject P is inserted. The rotating frame 17 provided in the cover 18 is disposed inside the cover 18, surrounding the central opening 19. The rotating frame 17 rotates around the central opening 19.

The X-ray CT device 1 is, for example, a rotate/rotate-type X-ray CT device (third generation CT) in which both the X-ray tube 11 and the X-ray detector 15 are supported by the rotating frame 17 and rotate around the subject P, but is not limited thereto and may be a stationary/rotate-type X-ray CT device (fourth generation CT) in which a plurality of X-ray detection elements arranged in a circular ring are fixed to a fixed frame and the X-ray tube 11 rotates around the subject P.

The gantry driving device 22 includes, for example, a base 101, a horizontal movement device 102, a support column 103, a rail 104, a slider 105, and a tilt mechanism 106. The base 101 includes, for example, a linear support structure extending in the horizontal direction. For example, the base 101 is fixed to the floor surface in an examination room by bolts or the like.

The horizontal movement device 102 is provided on the base 101, and the support column 103 is mounted on the horizontal movement device 102 in an upright state. The support column 103 is, for example, a member extending in the vertical direction. The horizontal movement device 102 moves the support column 103 mounted thereon in the horizontal direction on the basis of control of the control device 24.

The rail 104 is attached to the support column 103. The rail 104 is disposed in a direction (vertical direction) in which the support column 103 extends. The slider 105 is attached to the rail 104. The slider 105 is movable along the rail 104 on the basis of control of the control device 24.

The tilt mechanism 106 is attached to the slider 105, and the gantry 20 is attached to the tilt mechanism 106. The tilt mechanism 106 is capable of tilting the gantry 20 around a rotation axis on the basis of control of the control device 24. The tilt mechanism 106 changes the orientation of the gantry 20 by tilting the gantry 20. The gantry driving device 22 moves the gantry 20 in the horizontal direction by moving the support column 103 according to the horizontal movement device 102.

The gantry driving device 22 moves the gantry 20 in the vertical direction by moving the slider 105 along the rail 104. The gantry driving device 22 tilts the gantry 20 around the rotation axis with the tilt mechanism 106. The gantry driving device 22 moves the gantry 20 and the subject P relatively by moving the gantry 20.

The control device 24 includes processing circuitry having a processor such as a central processing unit (CPU) and a drive mechanism including a motor, an actuator, and the like. The processing circuitry realizes these functions by, for example, a hardware processor executing a program stored in a storage device (storage circuit).

The hardware processor refers to circuitry such as a CPU, a graphics processing unit (GPU), an application specific integrated circuit (ASIC), a programmable logic device (for example, a simple programmable logic device (SPLD) or a complex programmable logic device (CPLD)), and a field programmable gate array (FPGA). Instead of storing a program in a storage device, the program may be directly built into the circuitry of the hardware processor. In this case, the hardware processor realizes functions by reading and executing the program built into the circuitry. The hardware processor is not limited to being configured as single circuitry, but may be configured as a single hardware processor by combining a plurality of independent circuits to realize each function. The storage device may be a non-transitory (hardware) storage medium. Further, a plurality of components may be integrated into a single hardware processor to realize each function.

For example, the control device 24 rotates the rotating frame 17, moves the gantry 20 through the gantry driving device 22, and moves the top plate 33 of the bed device 30. The top plate 33 serves as a bed on which the subject P is placed in a lying position. For example, when tilting the gantry 20, the control device 24 controls the tilt mechanism 106 of the gantry driving device 22 to rotate the rotating frame 17 around an axis parallel to the Z-axis direction on the basis of an inclination angle (tilt angle) input to an input interface 43.

The control device 24 ascertains the rotation angle of the rotating frame 17 from the output of a sensor (not shown) or the like. Further, the control device 24 also provides the rotation angle of the rotating frame 17 to the processing circuitry 50 at any time. The control device 24 may be provided in the gantry 10 or in the console device 40. The control device 24 moves the gantry 20 along the rails 104 to perform main scan imaging or to perform scan imaging of capturing a scan image which is a positioning image performed before main scan imaging. The scan image is assumed to be an image captured from the side of the subject P, for example. The control device 24 outputs the scan image to a trajectory setting function 55 of the console device 40.

The control device 24 controls the gantry driving device 22 to move the gantry 20 along a predetermined trajectory, and causes the X-ray detector 15 to detect X-rays radiated by the X-ray tube 11 to scan the subject P. The predetermined trajectory is, for example, a trajectory set by the trajectory setting function 55, which will be described later.

The bed device 30 is a device that places the subject P to be scanned thereon, move the subject P and introduces the subject P into the rotating frame 17 of the gantry 10. The bed device 30 includes, for example, a base 31, a bed driving device 32, a top plate 33, and a support frame 34.

The base 31 includes a housing that supports the support frame 34 such that the support frame 34 can move in the vertical direction (Y-axis direction). The bed driving device 32 includes a motor and an actuator. The bed driving device 32 moves the top plate 33 on which the subject P is placed along the support frame 34 in the longitudinal direction of the top plate 33 (Z-axis direction). The top plate 33 is a plate-shaped member on which the subject P is placed. The bed driving device 32 moves the top plate 33 backward and inserts the same into the opening of the gantry 20. The bed driving device 32 moves the top plate 33 forward and pulls the same out from the gantry 20.

The bed driving device 32 may move not only the top plate 33 but also the support frame 34 in the longitudinal direction of the top plate 33. Contrary to the above, the gantry 10 may be movable in the Z-axis direction, and the gantry 10 may be moved to control the rotating frame 17 to come around the subject P. Further, both the gantry 10 and the top plate 33 may be configured to be movable.

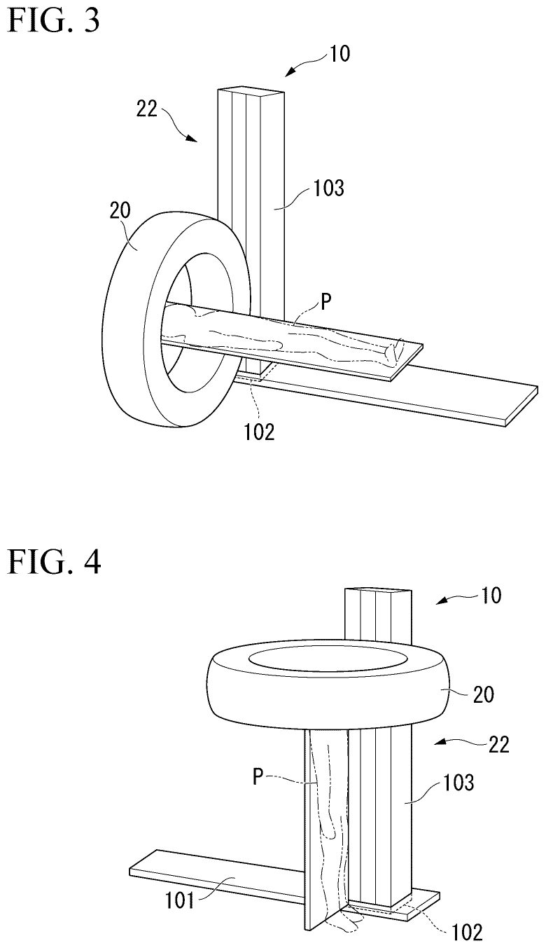

FIG. 3 is a perspective view of the X-ray CT device 1 that examines a subject P in a lying position. FIG. 4 is a perspective view of the X-ray CT device 1 that examines a subject P in a standing position. The subject P placed on the bed device 30 is examined in a lying position as shown in FIG. 3. The X-ray CT device 1 can also examine a subject P in a standing position as shown in FIG. 4. When examining the subject P in a standing position, for example, a support device or the like that supports the subject P in a standing position is used. The bed device 30 may be equipped with a displacement structure that displaces the subject P between a lying position and a standing position, for example.

The gantry 10 and bed device 30 change the relative movement direction of the X-ray detector 15 provided on the gantry 20 and the subject P supported on the top plate 33, for example, by using the horizontal movement device 102, the slider 105, and the bed driving device 32. For example, when scanning the entire body of the subject P disposed parallel to the Z direction, the gantry 20 is moved in the Z direction together with the support column 103 by the horizontal movement device 102. When scanning the subject P along an OM line tilted from the Z direction around the X-axis, the gantry 20 is moved in the Z direction and Y direction by the horizontal movement device 102 and the slider 105.

The console device 40 includes, for example, a memory 41, a display 42, an input interface 43, and processing circuitry 50. In the embodiment, the console device 40 is described as being separate from the gantry 10, but the gantry 10 may include some or all of the components of the console device 40.

The memory 41 is realized, for example, by a semiconductor memory element such as a random access memory (RAM) or a flash memory, a hard disk, an optical disk, and the like. The memory 41 stores, for example, detection data, projection data, reconstructed image data, and CT image data. Such data may be stored in an external memory (not shown) with which the X-ray CT device 1 can communicate, instead of the memory 41 (or in addition to the memory 41). The external memory is controlled by a cloud server that manages the external memory, for example, by receiving a read/write request.

The display 42 displays various types of information. For example, the display 42 is an output interface that displays medical images (CT images) generated by the processing circuitry, graphical user interface (GUI) images for receiving various operations of an operator such as a doctor or an engineer, and the like. The display 42 is, for example, a liquid crystal display (LCD), a cathode ray tube (CRT), an organic electroluminescence (EL) display, or the like. The display 42 may be provided on the gantry 10. The display 42 may be a desktop type, or a display device (e.g., a tablet terminal) that can wirelessly communicate with the main body of the console device 40.

The input interface 43 receives various input operations of an operator and outputs an electrical signal indicating the content of a received input operation to the processing circuitry 50. For example, the input interface 43 receives input operations such as collection conditions at the time of collecting detection data or projection data, reconstruction conditions at the time of reconstructing a CT image, and image processing conditions at the time of generating a post-processed image from a CT image. The input interface 43 is realized by, for example, a mouse, a keyboard, a touch panel, a track ball, a switch, a button, a joystick, a camera, an infrared sensor, a microphone, etc. The input interface 43 may be realized by a display device (e.g., a tablet terminal) capable of wireless communication with the main body of the console device 40. The input interface 43 outputs an electrical signal for setting a scan trajectory to the processing circuitry 50 on the basis of an operation of the operator.

In this specification, the input interface is not limited to an input interface having physical operating parts such as a mouse and a keyboard. For example, examples of the input interface may also include electrical signal processing circuitry that receives an electrical signal corresponding to an input operation from external input equipment provided separately from the apparatus and outputs the electrical signal to a control circuit.

The processing circuitry 50 controls the overall operation of the X-ray CT device 1. The processing circuitry 50 includes, for example, a control function 51, a preprocessing function 52, a reconstruction processing function 53, an image processing function 54, and a trajectory setting function 55. The processing circuitry 50 realizes these functions by, for example, a hardware processor executing a program stored in a storage device (storage circuit).

The hardware processor refers to circuitry such as a CPU, a GPU, an application specific integrated circuit, a programmable logic device, a complex programmable logic device, or a field programmable gate array. Instead of storing the program in a storage device, the program may be directly incorporated into the circuit of the hardware processor. The hardware processor is not limited to being configured as a single circuit, but may be configured as a single hardware processor by combining a plurality of independent circuits to realize each function. The storage device may be a non-transitory (hardware) storage medium. Further, a plurality of components may be integrated into a single hardware processor to realize each function.

The components of the console device 40 or the processing circuitry 50 may be distributed and realized by a plurality of pieces of hardware. The processing circuitry 50 may be realized by a processing device capable of communicating with the console device 40, rather than being a component included in the console device 40. The processing device is, for example, a workstation connected to one X-ray CT device, or a device (e.g., a cloud server) connected to a plurality of X-ray CT devices and collectively executing processing equivalent to that of the processing circuitry 50 which will be described below.

The control function 51 controls various functions of the processing circuitry 50 on the basis of input operations received through the input interface 43. For example, the control function 51 executes processing of collecting detection data in the gantry 10, and the like by controlling the X-ray high voltage device 14, the DAS 16, the control device 24, and the bed driving device 32 of the bed device 30.

The preprocessing function 52 performs preprocessing such as logarithmic conversion processing, offset correction processing, inter-channel sensitivity correction processing, and beam hardening correction on detection data output by the DAS 16, generates projection data, and stores the generated projection data in the memory 41.

The reconstruction processing function 53 performs reconstruction processing according to filtered back projection, iterative reconstruction, or the like on projection data set by the preprocessing function 52 to generate CT image data, and stores the generated CT image data in the memory 41.

The image processing function 54 converts CT image data into three-dimensional image data or cross-sectional image data of an arbitrary cross section by a known method on the basis of an input operation received through the input interface 43. Conversion into three-dimensional image data may be performed by the preprocessing function 52.

The trajectory setting function 55 receives setting information for setting a trajectory on the basis of an input operation of the operator received through the input interface 43. The trajectory setting function 55 sets a scan trajectory for scanning the subject P on the basis of the input setting information.

When setting a scan trajectory, the trajectory setting function 55 displays a trajectory setting image including an image showing the outer shape of the subject P on the display 42. The trajectory setting function 55 acquires a scan image output by the control device 24. The trajectory setting function 55 displays the scan image on the display 42 and sets a scan trajectory. Note that the X-ray CT device 1 does not need to have the trajectory setting function 55.

Hereinafter, a method of generating a reconstructed image corrected to an arbitrary orientation by the X-ray CT device 1 of the embodiment will be described.

As described above, the X-ray CT device 1 of the embodiment is a universal X-ray CT device that can capture images of subjects in a standing or sitting position, for example. When capturing images in a standing or sitting position, a subject can easily change the orientation of his/her body, and thus a deviation is likely to occur between the orientation of the body of the subject in a reconstructed image and the direction of the reference position. When such a deviation occurs, diagnosis such as interpretation may become difficult. In addition, when such a deviation is corrected by performing image processing to rotate the orientation of the reconstructed image, image noise such as blurred contours may occur depending on the rotation angle. In addition, such image processing increases the time required for image reconstruction. Furthermore, when noise reduction processing using AI is performed, noise reduction effect may decrease if a reconstructed image in which the origination of the body of the subject is different from a predetermined direction is input.

For such a problem, the X-ray CT device 1 of the embodiment corrects the reference position of the X-ray tube before reconstructing an image, and therefore can generate a reconstructed image corrected in any orientation without degrading the image quality of the reconstructed image. In addition, the X-ray CT device 1 of the embodiment does not require image processing such as image rotation, and therefore can curb an increase in the time required for image reconstruction. Furthermore, since the X-ray CT device 1 of the embodiment can generate a reconstructed image corrected in any orientation, it is possible to prevent a decrease in the noise reduction effect when performing noise reduction processing using AI.

A specific configuration is as follows. In the case of a conventional X-ray CT device, an image is generally reconstructed such that the upward direction of the gantry (i.e., the 0-degrees direction of a rotating part inside the gantry) becomes the upward direction of a reconstructed image. In contrast, the X-ray CT device 1 of the embodiment performs correction such that a desired direction in the gantry 20 becomes the reference position of the X-ray tube 11 before reconstructing an image. Then, the X-ray CT device 1 reconstructs an image such that the direction of the corrected reference position becomes the upward direction of the reconstructed image.

Four embodiments of correction processing for correcting the reference position of the X-ray tube 11 in a desired direction will be described below. In a first embodiment which will be described below, correction processing for physically correcting the reference position of the X-ray tube 11 by starting collection of detection data at the timing when the rotation angle of the rotating frame 17 has become a desired angle will be described. In second to fourth embodiments which will be described below, correction processing for correcting the reference position of the X-ray tube 11 on detection data by correcting the detection data used to generate a reconstructed image will be described. In the second embodiment, detection data is corrected on the side of the gantry 10 when the detection data is collected. In the third embodiment, detection data is corrected on the side of the console device 40 when the detection data is saved. In the fourth embodiment, detection data is corrected on the side of the console device 40 when an image is preprocessed for reconstruction.

A method of correcting the reference position of the X-ray tube 11 is not limited to methods according to the correction processing of the four embodiments, and other methods may be used.

Hereinafter, the correction processing of the first embodiment will be described.

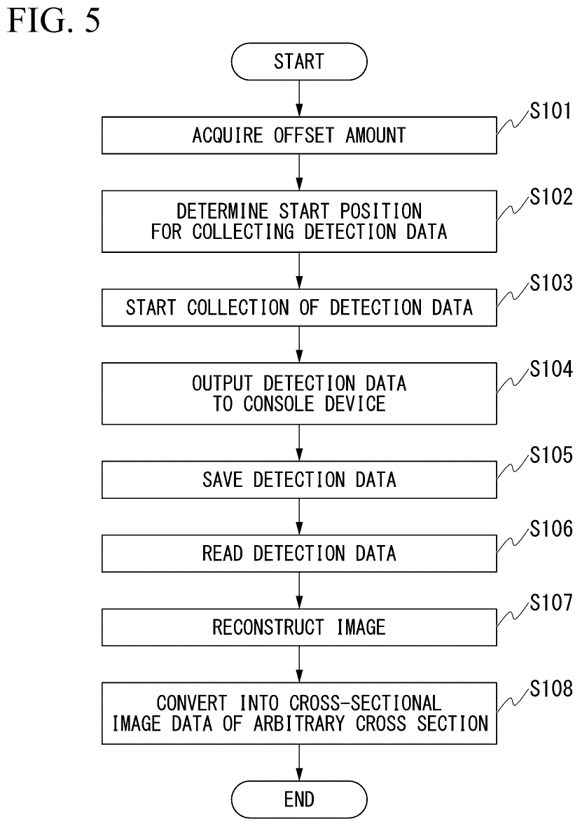

The X-ray CT device 1 in the first embodiment has a configuration in which collection of detection data is started at the timing when the rotation angle of the rotating frame 17 has become a desired angle. FIG. 5 is a flowchart showing an operation of the X-ray CT device 1 in the first embodiment.

The control function 51 of the console device 40 acquires information indicating an offset amount (step S101). The offset amount mentioned here is information indicating the magnitude of deviation between the orientation of the initial reference position of the X-ray tube 11 and the orientation of a subject. The orientation of the initial reference position of the X-ray tube 11 is, for example, the upward direction of the gantry 20 (0-degrees direction of the rotating frame 17 inside the gantry), which is a reference position in a conventional X-ray CT device. However, the orientation of the initial reference position of the X-ray tube 11 may be any predetermined direction.

The control function 51 acquires the information indicating the offset amount, for example, from the input interface 43. In this case, information indicating the offset amount generated on the basis of an input operation of the operator received through the input interface 43 is input to the control function 51. For example, a subject standing or sitting under the gantry 20 is displayed on the display 42. The operator identifies a required offset amount by visually checking the orientation of the body of the subject displayed on the display 42. The operator operates the input interface 43 to input the identified offset amount.

The operator may manually rotate the image such that the orientation of the initial reference position of the X-ray tube 11 matches the orientation of the body of the subject displayed on the display 42 and identify the offset amount from the amount of rotation.

The X-ray CT device 1 or a sensor (not shown) installed near the X-ray CT device 1 may automatically detect the orientation of the body of the subject and output information indicating the offset amount to the control function 51. In this case, for example, the orientation of the body of the subject may be detected on the basis of the longitudinal direction and the lateral direction of a cross-sectional image of the body of the subject. In this manner, any method can be used to identify a required offset amount.

The control function 51 determines a start position for collecting detection data on the basis of the acquired information indicating the offset amount (step S102). As an example, it is assumed that a reconstructed image is generated with the start position for collecting detection data as a reference position. For example, a plurality of sensors (not shown) are installed inside the gantry 20 at a predetermined interval along a trajectory of the X-ray tube 11 that moves as the rotating frame 17 rotates. As the interval between the plurality of sensors decreases (i.e., the number of sensors installed increases), the deviation can be corrected more accurately. These sensors detect the X-ray tube 11 passing in front of the device. The control function 51 starts collection of detection data by the X-ray tube 11, the X-ray detector 15, and the DAS 16 at the timing when the sensor installed near the determined start position for collecting detection data detects passing of the X-ray tube 11 (step S103). This allows actual collection of detection data to be started at the determined start position for collecting detection data.

The DAS 16 collects detection data for the entire circumference (360 degrees) and outputs collected detection data group to the console device 40 (step S104). The console device 40 saves the detection data output from the DAS 16 in the memory 41 (step S105). Thereafter, the console device 40 waits until a timing at which a reconstructed image is generated, for example.

The preprocessing function 52 reads the detection data saved in the memory 41 (step S106). The preprocessing function 52 performs preprocessing such as logarithmic conversion processing, offset correction processing, inter-channel sensitivity correction processing, and beam hardening correction on the read detection data to generate projection data. The reconstruction processing function 53 performs reconstruction processing according to filtered back projection, iterative approximation reconstruction, or the like on a plurality of pieces of projection data generated by the preprocessing function 52 to generate reconstructed image data (CT image data) (step S107).

The image processing function 54 converts the CT image data into cross-sectional image data of an arbitrary cross section by a known method on the basis of an input operation received through the input interface 43 (step S108). The image processing function 54 may convert the CT image data into three-dimensional image data by a known method. This completes the operation of the X-ray CT device 1 of the first embodiment shown in the flowchart of FIG. 5.

As described above, the X-ray CT device 1 in the first embodiment determines the start position for collecting detection data in accordance with a deviation between the orientation of the subject and the reference position. Then, the X-ray CT device 1 in the first embodiment generates projection data from the collected detection data using the determined position as a reference position, and generates a reconstructed image. In this manner, the X-ray CT device 1 in the first embodiment has a configuration in which the reference position is corrected in advance before a reconstructed image is generated, and thus a reconstructed image in a desired orientation can be easily generated without the need for subsequent image processing such as image rotation.

Hereinafter, the correction processing of the second embodiment will be described.

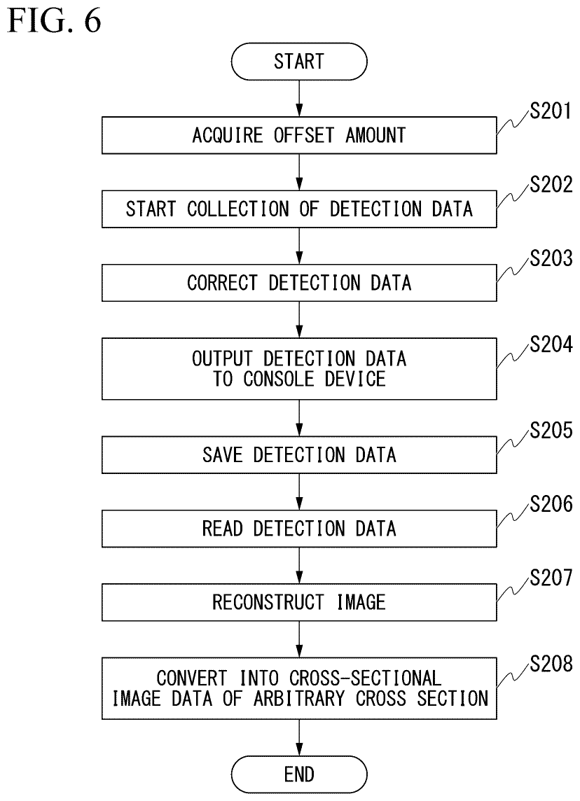

The X-ray CT device 1 in the second embodiment has a configuration in which the reference position of the X-ray tube 11 is corrected on detection data used to generate a reconstructed image by correcting the detection data. In the second embodiment, detection data is corrected on the side of the gantry 10 when the detection data is collected. FIG. 6 is a flowchart showing an operation of the X-ray CT device 1 in the second embodiment.

The DAS 16 of the gantry 10 acquires information indicating an offset amount (step S201). As described above, the offset amount is angle information indicating a deviation between the orientation of the initial reference position of the X-ray tube 11 and the orientation of a subject. The orientation of the initial reference position of the X-ray tube 11 is, for example, the upward direction of the gantry 20 (0-degrees direction of the rotating frame 17 inside the gantry), which is a reference position in a conventional X-ray CT device. However, the orientation of the initial reference position of the X-ray tube 11 may be any predetermined direction.

The DAS 16 acquires the information indicating the offset amount, for example, from the input interface 43. In this case, information indicating the offset amount generated on the basis of an input operation of the operator received through the input interface 43 is input to the DAS 16. The X-ray CT device 1 or a sensor (not shown) installed near the X-ray CT device 1 may detect the orientation of the body of the subject and output information indicating the offset amount to the DAS 16.

The control function 51 causes the X-ray tube 11, the X-ray detector 15, and the DAS 16 to start collection of detection data (step S202). The DAS 16 collects detection data for the entire circumference (360 degrees). The DAS 16 corrects the reference position of the X-ray tube 11 by correcting a collected detection data group (step S203).

Specifically, the DAS 16 corrects, for example, a view number included in the detection data. As described above, the view number is a number that changes according to rotation of the rotating frame 17, for example, a number that is incremented according to rotation of the rotating frame 17. Therefore, the view number is information indicating the rotation angle of the X-ray tube 11. The DAS 16 corrects the view number on the basis of the acquired information indicating the offset amount. For example, if the value of the view number is the value of the rotation angle itself from the initial reference position of the X-ray tube 11, the DAS 16 performs correction to increment the value of the view number by the value of the offset amount.

The DAS 16 outputs the corrected detection data group to the console device 40 (step S204). The console device 40 saves the detection data output from the DAS 16 in the memory 41 (step S205). Thereafter, the console device 40 waits until, for example, a timing when a reconstructed image is generated.

The preprocessing function 52 reads the detection data saved in the memory 41 (step S206). The preprocessing function 52 performs preprocessing such as logarithmic conversion processing, offset correction processing, inter-channel sensitivity correction processing, and beam hardening correction on the read detection data to generate projection data. The reconstruction processing function 53 performs reconstruction processing according to filtered back projection, iterative approximation reconstruction, or the like on a plurality of pieces of projection data generated by the preprocessing function 52 to generate reconstructed image data (CT image data) (step S207).

The image processing function 54 converts the CT image data into cross-sectional image data of an arbitrary cross section by a known method on the basis of an input operation received through the input interface 43 (step S208). The image processing function 54 may convert the CT image data into three-dimensional image data by a known method. This completes the operation of the X-ray CT device 1 of the second embodiment shown in the flowchart of FIG. 6.

As described above, the X-ray CT device 1 in the second embodiment corrects the view number included in the detection data in accordance with the deviation between the orientation of the subject and the reference position. Then, the X-ray CT device 1 in the second embodiment generates projection data from collected detection data on the basis of the corrected view number, and generates a reconstructed image. In this manner, the X-ray CT device 1 in the second embodiment has a configuration in which the reference position is corrected by changing the view number included in the detection data before a reconstructed image is generated, and thus a reconstructed image in a desired orientation can be easily generated without requiring subsequent image processing such as image rotation.

Hereinafter, the correction processing of the third embodiment will be described.

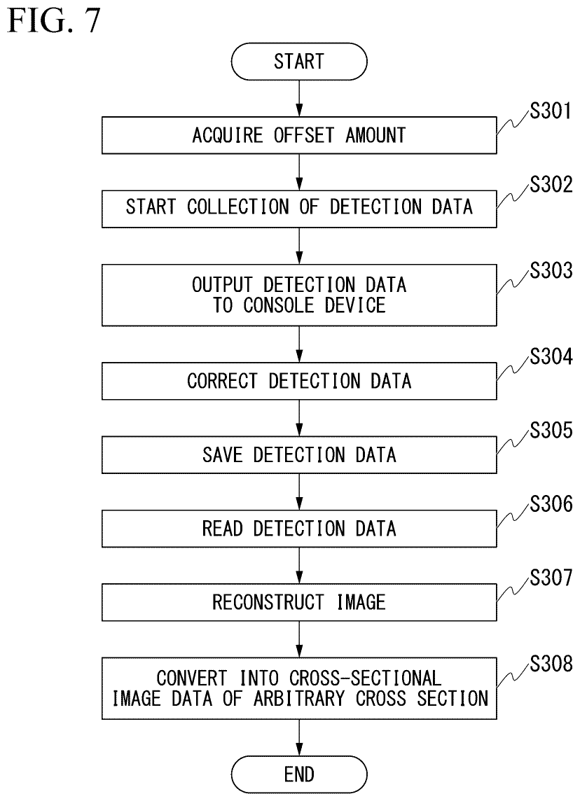

The X-ray CT device 1 in the third embodiment has a configuration in which the reference position of the X-ray tube 11 is corrected on detection data used to generate a reconstructed image by correcting the detection data. In the third embodiment, detection data is corrected on the side of the console device 40 when the detection data is saved. FIG. 7 is a flowchart showing an operation of the X-ray CT device 1 in the third embodiment.

The control function 51 of the console device 40 acquires information indicating an offset amount (step S301). As described above, the offset amount is angle information indicating a deviation between the orientation of the initial reference position of the X-ray tube 11 and the orientation of a subject. The orientation of the initial reference position of the X-ray tube 11 is, for example, the upward direction of the gantry 20 (0-degrees direction of the rotating frame 17 inside the gantry), which is a reference position of a conventional X-ray CT device. However, the orientation of the initial reference position of the X-ray tube 11 may be any predetermined direction.

The control function 51 acquires information indicating the offset amount from, for example, the input interface 43. In this case, information indicating the offset amount generated on the basis of an input operation of the operator received through the input interface 43 is input to the control function 51. The X-ray CT device 1 or a sensor (not shown) installed near the X-ray CT device 1 may detect the orientation of the body of the subject and output information indicating the offset amount to the DAS 16.

The control function 51 causes the X-ray tube 11, the X-ray detector 15, and the DAS 16 to start collection of detection data (step S302). The DAS 16 of the gantry 10 collects detection data for the entire circumference (360 degrees) and outputs a collected detection data group to the console device 40 (step S303). The control function 51 corrects the detection data group output from the DAS 16 to correct the reference position of the X-ray tube 11 (step S304).

The control function 51 corrects a view number included in the detection data, for example, as described above. As described above, the view number is a number that changes according to rotation of the rotating frame 17, for example, a number that is incremented according to rotation of the rotating frame 17. The control function 51 corrects the view number on the basis of the acquired information indicating the offset amount. The control function 51 saves the corrected detection data group in the memory 41 (step S305). Thereafter, the console device 40 waits until a timing when a reconstructed image is generated, for example.

The preprocessing function 52 reads the detection data saved in the memory 41 (step S306). The preprocessing function 52 performs preprocessing such as logarithmic conversion processing, offset correction processing, inter-channel sensitivity correction processing, and beam hardening correction on the read detection data, and generates projection data. The reconstruction processing function 53 performs reconstruction processing according to filtered back projection, iterative reconstruction, or the like, on a plurality of pieces of projection data generated by the preprocessing function 52 to generate reconstructed image data (CT image data) (step S307).

The image processing function 54 converts the CT image data into cross-sectional image data of an arbitrary cross section by a known method on the basis of an input operation received through the input interface 43 (step S308). The image processing function 54 may convert the CT image data into three-dimensional image data by a known method. This completes the operation of the X-ray CT device 1 of the third embodiment shown in the flowchart of FIG. 7.

As described above, the X-ray CT device 1 in the third embodiment corrects the view number included in the detection data in accordance with the deviation between the orientation of the subject and the reference position. Then, the X-ray CT device 1 in the third embodiment generates projection data from the collected detection data on the basis of the corrected view number, and generates a reconstructed image. In this manner, the X-ray CT device 1 of the second embodiment has a configuration in which the reference position is corrected by changing the view number included in the detection data before a reconstructed image is generated, and thus a reconstructed image in a desired orientation can be easily generated without requiring subsequent image processing such as image rotation.

Hereinafter, the correction processing of the fourth embodiment will be described.

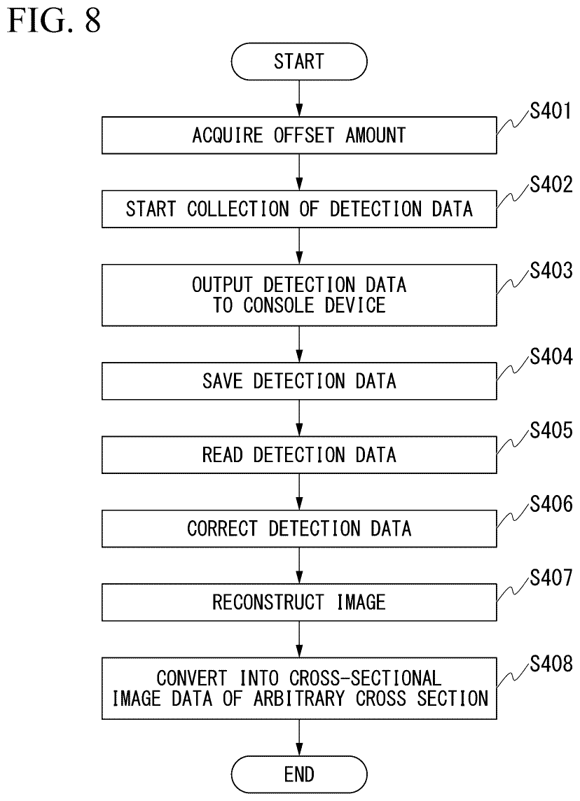

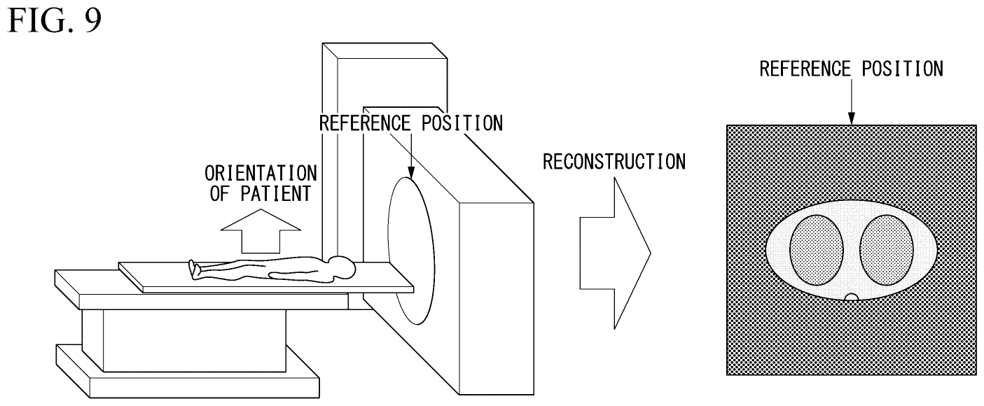

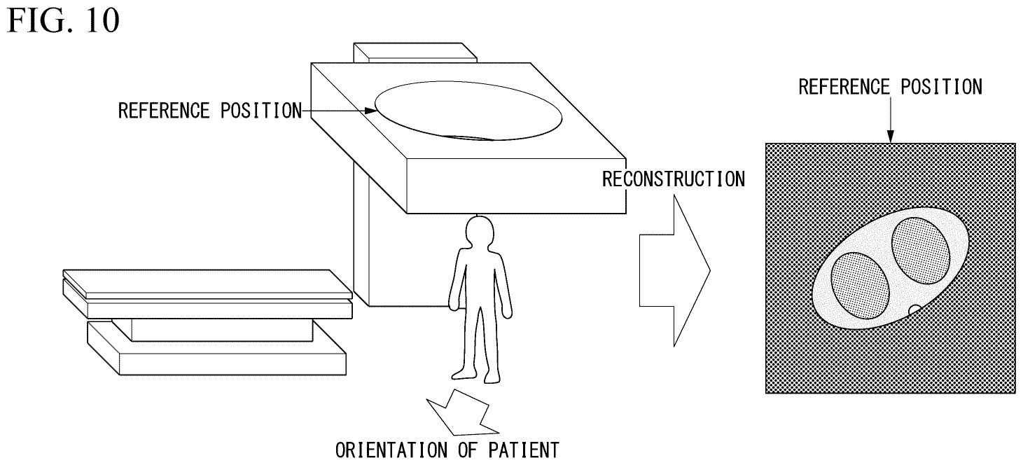

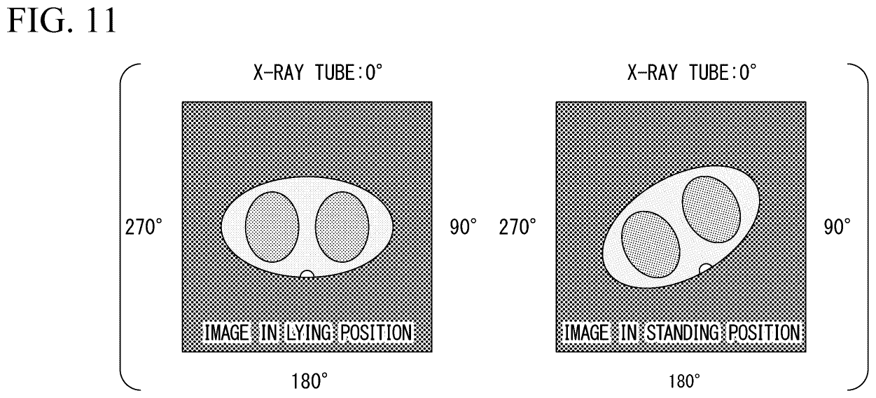

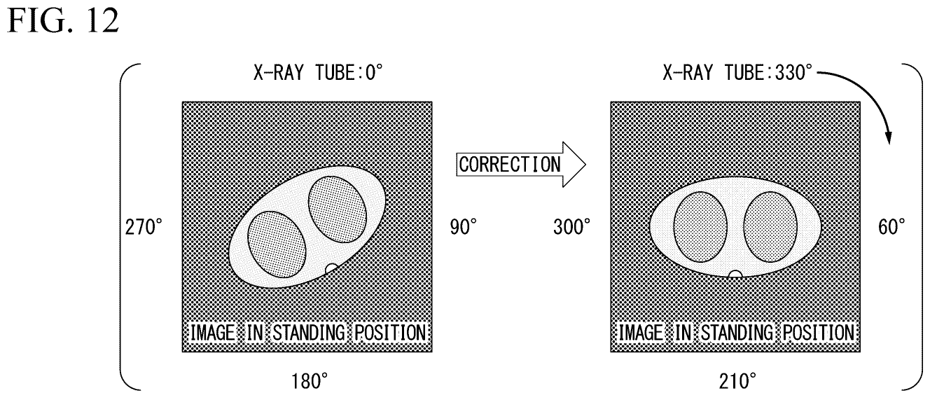

The X-ray CT device 1 in the fourth embodiment has a configuration in which the reference position of the X-ray tube 11 is corrected on detection data used to generate a reconstructed image by correcting the detection data. In the fourth embodiment, detection data is corrected on the side of the console device 40 when an image is preprocessed for reconstruction. FIG. 8 is a flowchart showing an operation of the X-ray CT device 1 in the fourth embodiment.

The control function 51 of the console device 40 acquires information indicating an offset amount (step S401). As described above, the offset amount is angle information indicating a deviation between the orientation of the initial reference position of the X-ray tube 11 and the orientation of a subject. The orientation of the initial reference position of the X-ray tube 11 is, for example, the upward direction of the gantry 20 (0-degrees direction of the rotating frame 17 inside the gantry), which is a reference position in a conventional X-ray CT device. However, the orientation of the initial reference position of the X-ray tube 11 may be any predetermined direction.

The control function 51 acquires information indicating the offset amount, for example, from the input interface 43. In this case, information indicating the offset amount generated on the basis of an input operation of the operator received through the input interface 43 is input to the control function 51. The X-ray CT device 1 or a sensor (not shown) installed near the X-ray CT device 1 may detect the orientation of the body of the subject and output information indicating the offset amount to the DAS 16.

The control function 51 causes the X-ray tube 11, the X-ray detector 15, and the DAS 16 to start collection of detection data (step S402). The DAS 16 of the gantry 10 collects detection data for the entire circumference (360 degrees) and outputs a collected detection data group to the console device 40 (step S403). The console device 40 saves the detection data output from the DAS 16 in the memory 41 (step S404). Thereafter, the console device 40 waits until a timing when a reconstructed image is generated, for example.

The preprocessing function 52 reads the detection data saved in the memory 41 (step S405). The preprocessing function 52 corrects the read detection data group to correct the reference position of the X-ray tube 11 (step S406).

The preprocessing function 52 corrects a view number included in the detection data, for example, as described above. As described above, the view number is a number that changes according to rotation of the rotating frame 17, for example, a number that is incremented according to rotation of the rotating frame 17. The preprocessing function 52 corrects the view number on the basis of the acquired information indicating the offset amount.

The preprocessing function 52 performs preprocessing such as logarithmic conversion processing, offset correction processing, inter-channel sensitivity correction processing, and beam hardening correction on the corrected detection data to generate projection data. The reconstruction processing function 53 performs reconstruction processing according to filtered back projection, iterative reconstruction, or the like on a plurality of pieces of projection data generated by the preprocessing function 52 to generate reconstructed image data (CT image data) (step S407).

The image processing function 54 converts the CT image data into cross-sectional image data of an arbitrary cross section by a known method on the basis of an input operation received through the input interface 43 (step S408). The image processing function 54 may convert the CT image data into three-dimensional image data by a known method. This completes the operation of the X-ray CT device 1 of the fourth embodiment shown in the flowchart of FIG. 8.

As described above, the X-ray CT device 1 in the fourth embodiment corrects the view number included in the detection data in accordance with the deviation between the orientation of the subject and the reference position. Then, the X-ray CT device 1 in the fourth embodiment generates projection data from the collected detection data on the basis of the corrected view number, and generates a reconstructed image. In this manner, the X-ray CT device 1 in the fourth embodiment has a configuration in which the reference position is corrected by changing the view number included in the detection data before a reconstructed image is generated, and thus a reconstructed image in a desired orientation can be easily generated without the need for subsequent image processing such as image rotation.

Although the gantry 20 is moved when the gantry 20 and the subject P are moved relative to each other in the X-ray CT device 1 of the above embodiment, instead of moving the gantry 20, the top plate 33 of the bed device 30 may be moved. Alternatively, both the gantry 20 and the top plate 33 may be moved.

Although no adjustment is performed using the wedge 12 or the collimator 13 in the X-ray CT device 1 of the embodiment, for example, an active collimator may be used as the wedge 12 or the collimator 13, and unnecessary parts of projection data may be cut when executing either helical scanning or volume scanning as a scanning state. By cutting unnecessary parts of projection data using the collimator 13, the amount of X-ray exposure to the subject P during imaging using the X-ray CT device can be reduced. In addition, the amount of exposure may be reduced by making at least one of a helical pitch and a tube current of the X-ray tube 11 in each section suitable (optimum) for the subject.

According to at least one embodiment described above, the medical image diagnostic device includes an imaging unit that rotates around a subject, images the subject at a plurality of different positions including a predetermined reference position, and generates a plurality of pieces of raw image data including information indicating the predetermined reference position, a control unit that corrects the reference position depending on a deviation between an orientation of the subject and a direction from the subject to the predetermined reference position, and causes the imaging unit to image the subject based on the corrected reference position, and a reconstruction unit that generates a reconstructed image by reconstructing the plurality of pieces of raw image data generated by the imaging unit on the basis of the corrected reference position, thereby making it possible to easily generate a reconstructed image in which the subject faces in a desired direction without degrading image quality.

Further, according to at least one embodiment described above, by setting the reference position as a start position of imaging by the imaging unit, it is possible to specify both the reference position and the start position of imaging, thereby making it possible to simplify the operation and calculation processing for generating a reconstructed image.

Further, according to at least one embodiment described above, the medical image diagnostic device has an imaging unit that rotates around a subject, images the subject at a plurality of different positions including a predetermined reference position, and generates a plurality of pieces of raw image data including information indicating a relative position with respect to the predetermined reference position, a correction unit that corrects the information indicating the relative position included in the raw image data depending on a deviation between an orientation of the subject and a direction from the subject to the predetermined reference position, and a reconstruction unit that generates a reconstructed image by reconstructing the plurality of pieces of raw image data generated by the imaging unit on the basis of the corrected relative position corrected by the correction unit, thereby making it possible to easily generate a reconstructed image in which the subject faces a desired direction without degrading image quality.

Further, according to at least one embodiment described above, the information indicating the relative position is set as a view number that increases according to a rotation angle of the imaging unit, and thus a reconstructed image in which the subject faces a desired direction can be easily generated by simply correcting a view number of a conventional CT device. This also makes it possible to easily generate a reconstructed image by using a conventional calculation method as it is since there is no need to change the data configuration of detection data.

Furthermore, according to at least one of the embodiments described above, by setting the row image data as detection data including the view number and an X-ray intensity value, it is possible to correct the reference position before imaging, and thus it is possible to easily generate a reconstructed image without the need for image processing such as image rotation.

Furthermore, according to at least one of the embodiments described above, the imaging unit can be configured to include an X-ray tube, an X-ray detector that detects X-rays that are radiated by the X-ray tube and have passed through a subject, and a gantry having a rotation mechanism that rotates around the subject, on which the X-ray tube and the X-ray detector are installed at positions facing each other. That is, the present invention is applicable to general X-ray CT devices and universal X-ray CT devices. Note that the present invention is also applicable to other devices as long as they are devices that captures images while rotating an image-capturing unit around a subject.

Furthermore, according to at least one of the embodiments described above, it is possible to easily generate an image in a desired orientation (for example, an image in which the abdomen of a subject faces upward) without requiring additional post-processing even with an apparatus such as a universal X-ray CT device that is used by changing imaging in a lying position, imaging in a standing position, and imaging in a sitting position. As a result, the X-ray CT device 1 of the embodiment can be used in the same workflow as when using a conventional general X-ray CT device.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnostic device comprising:
an imaging unit configured to image a subject at a plurality of different positions including a predetermined reference position while rotating around the subject and to generate a plurality of pieces of raw image data including information indicating the predetermined reference position; and
processing circuitry configured to correct the reference position depending on a deviation between an orientation of the subject and a direction from the subject to the predetermined reference position, to cause the imaging unit to image the subject based on the corrected reference position, and to generate a reconstructed image by reconstructing the plurality of pieces of raw image data generated by the imaging unit based on the corrected reference position.

2. The medical image diagnostic device according to claim 1, wherein the reference position is a start position of imaging by the imaging unit.

3. A medical image diagnostic device comprising:
an imaging unit configured to image a subject at a plurality of different positions including a predetermined reference position while rotating around the subject and to generate a plurality of pieces of raw image data including information indicating a relative position with respect to the predetermined reference position; and
processing circuitry configured to correct the information indicating the relative position included in the raw image data depending on a deviation between an orientation of the subject and a direction from the subject to the predetermined reference position, and to generate a reconstructed image by reconstructing the plurality of pieces of raw image data generated by the imaging unit based on the corrected relative position.

4. The medical image diagnostic device according to claim 3, wherein the information indicating the relative position is a view number that increases depending on a rotation angle of the imaging unit.

5. The medical image diagnostic device according to claim 4, wherein the raw image data includes the view number and an X-ray intensity value.

6. The medical image diagnostic device according to claim 1, wherein the imaging unit comprises:
an X-ray tube;
an X-ray detector configured to detect X-rays radiated by the X-ray tube and having passed through the subject; and
a gantry having a rotation mechanism configured to rotate around the subject, on which the X-ray tube and the X-ray detector are installed at positions facing each other.

7. A method of controlling a medical image diagnostic device, using a computer of the medical image diagnostic device including an imaging unit configured to image a subject at a plurality of different positions including a predetermined reference position while rotating around the subject and to generate a plurality of pieces of raw image data including information indicating the predetermined reference position, comprising:
correcting the reference position depending on a deviation between an orientation of the subject and a direction from the subject to the predetermined reference position;
causing the imaging unit to image the subject based on the corrected reference position; and
generating a reconstructed image by reconstructing the plurality of pieces of raw image data generated by the imaging unit based on the corrected reference position.

8. A method of controlling a medical image diagnostic device, using a computer of the medial image diagnostic apparatus including an imaging unit configured to image a subject at a plurality of different positions including a predetermined reference position while rotating around the subject and to generate a plurality of pieces of raw image data including information indicating a relative position with respect to the predetermined reference position, comprising:
correcting the information indicating the relative position included in the raw image data depending on a deviation between an orientation of the subject and a direction from the subject to the predetermined reference position; and
generating a reconstructed image by reconstructing the plurality of pieces of raw image data generated by the imaging unit based on the corrected relative position.
